(19) United States
(12) Patent Application Publication
(10) Pub. No.: US 2026/0294343 A1
(43) Pub. Date: Oct. 1, 2026

(54) Viscoelastic Foam Sensor Mount

(71) Applicant: iRhythm Technologies, Inc., San Francisco, CA (US)

(72) Inventors: Herculaas Johannes Botha, Newark, CA (US); Monica C. Lin, San Francisco, CA (US)

(73) Assignee: iRhythm Technologies, Inc., San Francisco, CA (US)

(21) Appl. No.: 19/574,089

(22) Filed: Mar. 20, 2026

Related U.S. Application Data
(60) Provisional application No. 63/781,161, filed on Mar. 31, 2025.

(51) Int. Cl. A61B 5/00; A61B 5/024

(52) U.S. Cl. CPC A61B 5/6833; A61B 5/02416; A61B 2562/185

(57) ABSTRACT

A viscoelastic foam sensor mount is described and is implementable to provide consistent sensor contact between a sensor assembly and a skin surface of an individual. In an example, a sensor assembly includes an adhesive component configured to secure the sensor assembly to the skin surface. A foam material is contained within the adhesive component to be disposed between the adhesive component and the skin surface. The foam material, for instance, exhibits a substantially uniform stress response under variable compressive strain. A sensor housing region is disposed within the foam material and a sensor is positioned within the sensor housing region. The foam material is configured to compress between the adhesive component and the skin surface to provide a distributed force across a contact area to maintain contact of the sensor with the skin surface for a duration of an observation period.

### RELATED APPLICATIONS

This application claims priority to U.S. Provisional Application No. 63/781,161, filed Mar. 31, 2025, and titled “Viscoelastic Foam Sensor Mount,” which is hereby incorporated by reference in its entirety.

### BACKGROUND

Sensors for monitoring physiological parameters are often integrated into wearable devices for continuous health tracking. These devices utilize various sensor types to measure physiological metrics such as heart rate, blood oxygen saturation, electrical activity of the heart, and so forth. However, maintaining consistent sensor contact with the skin surface presents challenges, particularly for extended wear periods. For instance, traditional mounting methods frequently encounter issues such as motion artifacts, skin irritation, or poor pressure distribution across different body contours or sites. Additionally, conventional sensor assemblies exhibit limited ability to balance secure attachment with user comfort, which leads to issues such as reduced wear time, a degraded user experience, and/or compromised data quality. Accordingly, these limitations in sensor mounting technology impact accuracy and reliability of physiological measurements.

### BRIEF DESCRIPTION OF THE DRAWINGS

FIG. 1 is a block diagram of a non-limiting example of an environment that is operable to employ viscoelastic foam sensor mount techniques as described herein.

FIG. 2 depicts a non-limiting example of a monitoring device.

FIG. 3 illustrates an example of a sensor assembly attachable to a skin surface of an individual.

FIG. 4 illustrates a compression graph showing example compression characteristics of the foam material used in the sensor assembly.

FIG. 5 illustrates an example of different perimeter designs for a foam material in a sensor assembly.

FIG. 6 illustrates an example of an uncompressed state and a compressed state of a sensor assembly that includes multiple foam regions.

FIG. 7 illustrates examples of variable viscoelastic foam behavior over time.

FIG. 8 depicts a flow diagram of a method in an example implementation that is performable to apply a sensor assembly to a skin surface of an individual.

### DETAILED DESCRIPTION

Conventional sensor mounting techniques for physiological monitoring devices often struggle to maintain consistent skin contact while ensuring user comfort over extended wear periods. Accordingly, such techniques experience issues such as motion artifacts, skin irritation, and uneven pressure distribution across different body contours. These limitations lead to reduced wear time, compromised data quality, and an overall degraded user experience, particularly during physical activity or long-term monitoring scenarios, which offsets advantages provided by physiological monitoring modalities.

To overcome these limitations, a sensor assembly, e.g., a wearable device, is described that leverages a viscoelastic foam material to provide a distributed pressure interface between sensors and skin surfaces of individuals, such as to maintain consistent contact between the sensors and the skin surface while minimizing localized stress points that cause discomfort and motion artifacts in conventional sensor mounting systems. In an example, the sensor assembly includes an adhesive component configured to secure the sensor assembly to the skin surface, with the foam material disposed between the adhesive component and the skin surface. A sensor housing region is disposed within the foam material, and a sensor is positioned within this housing region. When the sensor assembly is applied to an individual, the foam material compresses between the adhesive component and the skin surface, which provides a distributed force across a contact area to maintain sensor contact with the skin surface throughout an observation period. In various examples, the foam material exhibits a substantially uniform stress response under variable compressive strain.

Accordingly, the foam maintains a substantially consistent pressure against the skin during motion and/or various compressive pressures against the assembly. For example, when an individual wearing the sensor assembly moves or changes position, the foam dynamically compresses and continues to apply consistent pressure such that the sensor maintains contact with the skin surface. In this way, the sensor assembly adapts to body movements and contours while maintaining a consistent sensor-to-skin interface, which improves measurement accuracy and reduces motion artifacts compared to conventional mounting systems.

In some implementations, the foam material is a viscoelastic polyurethane foam with a compression response that exhibits a semi-constant pressure region between a broad range of compressive strain, e.g., between 10% and 70% compressive strain. This allows the sensor assembly to adapt to different skin contours and movements while maintaining a consistent contact force. The foam material may further have a variable density along one or more dimensions, which can be tailored to optimize performance for particular sensor types, body locations, or patient characteristics. Additionally, the foam can be tinted or textured, such as to block a controlled amount of ambient light to enhance an accuracy of optical sensors such as photoplethysmography (PPG) sensors. In some examples, the foam material may have a non-circular perimeter, e.g., a “flower-like” or scalloped shape, configured to increase a perimeter-to-surface-area ratio relative to a circular configuration of equivalent area, such as to reduce normal forces of compression on the skin surface while maximizing sensor contact area and adhesion strength.

In some examples, the foam material includes multiple regions with different properties to accommodate various sensor components within the sensor assembly. For instance, the foam material may include different regions having variable densities, compressibilities, optical properties, geometries, etc. This configuration enables the foam material to apply different force and/or pressure profiles to different sensor components, such as to apply a first force to light-emitting diodes of a PPG sensor array and a second force to photodiodes of the PPG sensor array. Each foam region may be tailored in terms of density, compressibility, and other mechanical properties to optimize interaction between particular sensor components and the skin surface.

Accordingly, the techniques and components described herein provide various advantages over conventional sensor mounting systems. The viscoelastic foam material provides improved conformity to skin surfaces, which reduces motion artifacts and enhances measurement accuracy and thus can conserve computational resources otherwise expended attempting to process noisy data. The distributed force across the contact area further minimizes localized stress points, which reduces the risk of skin irritation and improves user comfort during extended wear. The semi-constant pressure characteristic of the foam allows for consistent sensor contact despite variations in skin tension or movement, supporting reliable data collection over time. Further, the ability to tailor properties of the foam material (e.g., density, thickness, color, shape, viscoelastic characteristics, etc.) enables optimization for different sensor types and measurement locations, offering a versatile solution for various monitoring applications.

In some aspects, the techniques described herein relate to a sensor assembly attachable to a skin surface of an individual, the sensor assembly including: an adhesive component configured to secure the sensor assembly to the skin surface; a foam material at least partially contained within the adhesive component to be disposed between the adhesive component and the skin surface, the foam material having a substantially uniform stress response under variable compressive strain; a sensor housing region disposed within the foam material; and a sensor positioned within the sensor housing region, the foam material configured to compress between the adhesive component and the skin surface to provide a distributed force across a contact area to maintain contact of the sensor with the skin surface for a duration of an observation period.

In some aspects, the techniques described herein relate to a sensor assembly, wherein the foam material is a viscoelastic polyurethane foam.

In some aspects, the techniques described herein relate to a sensor assembly, wherein a compression response of the foam material exhibits a semi-constant pressure region between 10% and 70% compressive strain.

In some aspects, the techniques described herein relate to a sensor assembly, wherein the adhesive component extends beyond a perimeter of the foam material to provide adhesion to the skin surface.

In some aspects, the techniques described herein relate to a sensor assembly, wherein the sensor includes one or more of an electrode, or an optical sensor configured for photoplethysmography, the optical sensor including one or more of a photodiode or a light-emitting diode.

In some aspects, the techniques described herein relate to a sensor assembly, wherein the adhesive component includes a film adhesive structure.

In some aspects, the techniques described herein relate to a sensor assembly, wherein the foam material has a thickness based in part on a type of sensor to control a force applied by the sensor to the skin surface.

In some aspects, the techniques described herein relate to a sensor assembly, wherein the foam material has a non-circular perimeter shape to increase a perimeter to surface area ratio relative to a circular configuration of the foam material of an equivalent area to reduce edge lifting or peel stresses.

In some aspects, the techniques described herein relate to a sensor assembly, wherein the observation period is at least seven days, and the foam material is configured to exhibit mechanical creep to continually reduce a force applied by the sensor assembly to the skin surface at a particular rate during the observation period.

In some aspects, the techniques described herein relate to a foam material for use in a sensor assembly attachable to a skin surface of an individual, the foam material including: a viscoelastic composition having a substantially uniform stress response under variable compressive strain; a sensor housing region disposed within the foam material and configured to receive a sensor; and a compression response that exhibits a semi-constant pressure region across a range of compressive strain, the foam material configured to compress between an attachment component of the sensor assembly and the skin surface to provide a distributed force across a contact area.

In some aspects, the techniques described herein relate to a foam material, wherein the foam material is a viscoelastic polyurethane foam and the semi-constant pressure region is between 10% and 70% compressive strain.

In some aspects, the techniques described herein relate to a foam material, wherein the foam material has a variable density along one or more dimensions of the foam material.

In some aspects, the techniques described herein relate to a foam material, wherein the variable density includes one or more of a gradient density, a layered density, a variable radial density, a patterned density, or an anisotropic density.

In some aspects, the techniques described herein relate to a foam material, wherein the foam material is tinted to block a controlled amount of ambient light.

In some aspects, the techniques described herein relate to a foam material, wherein the foam material has a non-circular perimeter shape to increase a perimeter to surface area ratio relative to a circular configuration of the foam material of an equivalent area to reduce one or more of edge lifting, peel stresses, or a normal force of compression on the skin surface relative to the circular configuration.

In some aspects, the techniques described herein relate to a sensor assembly attachable to a skin surface of an individual, the sensor assembly including: an attachment component configured to secure the sensor assembly to the skin surface; a foam material having a substantially uniform stress response under variable compressive strain that includes a first region having a first property and a second region having a second property different from the first property; and a sensor configured to be disposed adjacent to the skin surface, the foam material configured to compress between the attachment component and the skin surface to maintain contact of the sensor with the skin surface during an observation period.

In some aspects, the techniques described herein relate to a sensor assembly, wherein the first property and the second property include one or more variable densities, compressibilities, optical properties, or geometries.

In some aspects, the techniques described herein relate to a sensor assembly, wherein the sensor includes a photoplethysmography (PPG) sensor array including one or more light-emitting diodes and one or more photodiodes, and wherein the first region overlies the one or more light-emitting diodes and the second region overlies the one or more photodiodes.

In some aspects, the techniques described herein relate to a sensor assembly, wherein the first region applies a first force to the one or more light-emitting diodes when compressed and the second region applies a second force to the one or more photodiodes when compressed, the first force different than the second force.

In some aspects, the techniques described herein relate to a sensor assembly, wherein the attachment component includes one or more of an adhesive component, an armband, a strap, or an elastic band.

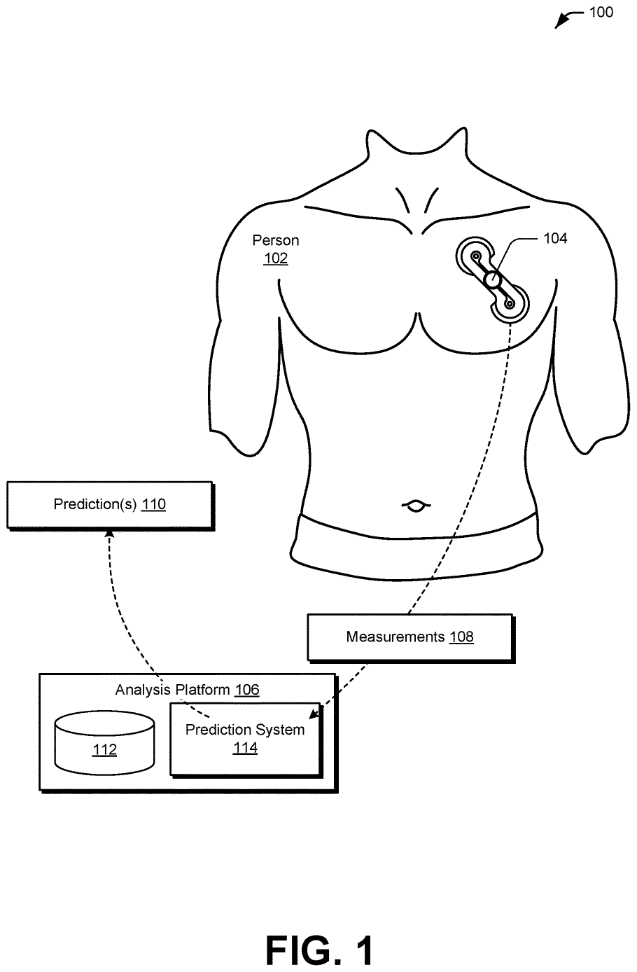

FIG. 1 is a block diagram of a non-limiting example 100 of an environment that is operable to employ viscoelastic foam sensor mount techniques as described herein. The illustrated example 100 includes a monitored subject, e.g., person 102, who is depicted wearing a monitoring device 104. The illustrated environment also includes an analysis platform 106. The analysis platform 106 may be connected to the monitoring device 104 via one or more wireless connections directly or via one or more wired and/or wireless connections and one or more intermediate devices, such as a computing device associated with the person 102, network routing devices and equipment, server devices, and/or the Internet, to name just a few.

The monitoring device 104 may be utilized to monitor one or more aspects of the person 102. By way of example, the monitoring device 104 may be utilized to monitor and/or be configured to perform one or more of electrocardiography (ECG), electroencephalography (EEG), electromyography (EMG), respiratory inductance plethysmography (RIP), pulse oximetry, accelerometry, impedance cardiography (ICG), or the like as measurements 108. As further described in more detail below, in some examples the monitoring device 104 of the illustrated example 100 includes a sensor assembly with a viscoelastic foam material for maintaining consistent sensor contact with the skin surface of the person 102. For instance, the sensor assembly may be utilized to consistently monitor one or more of heart rate, heart rate variability, blood oxygen saturation, respiration, electrical activity of the heart, and so forth.

In some scenarios, the monitoring device 104 may be provided to record measurements 108 of the person 102 over an observation period, e.g., lasting some number of seconds or minutes, lasting multiple days, and so on. By way of example, the electrical activity of the heart of the person 102 may be monitored over time to produce one or more electrocardiograms, which may be used to predict any of a variety of events. Alternatively, or in addition, the monitoring device 104 may be used to output the measurements 108 (e.g., a time sequence of measurements such as a time sequence of electric potential measurements), which may indicate an observation or be used to generate an assessment, diagnosis, or prediction of one or more events.

In connection with the monitoring device, instructions may be provided to the person 102 that instruct the person 102 how to operate the monitoring device 104 and/or how to behave (e.g., sleep, perform activity) while wearing the monitoring device 104. In one or more implementations, the instructions may be provided as part of a kit, e.g., written instructions. Alternatively or additionally, the analysis platform 106 may cause the instructions to be communicated to and output (e.g., for display and/or audio output) via a computing device associated with the person 102. In one or more implementations, the analysis platform 106 may wait to provide these instructions for output after a predetermined amount of time of an observation period has lapsed (e.g., two days) while wearing the monitoring device 104 and/or based on patterns in the aspects of the person 102 being measured.

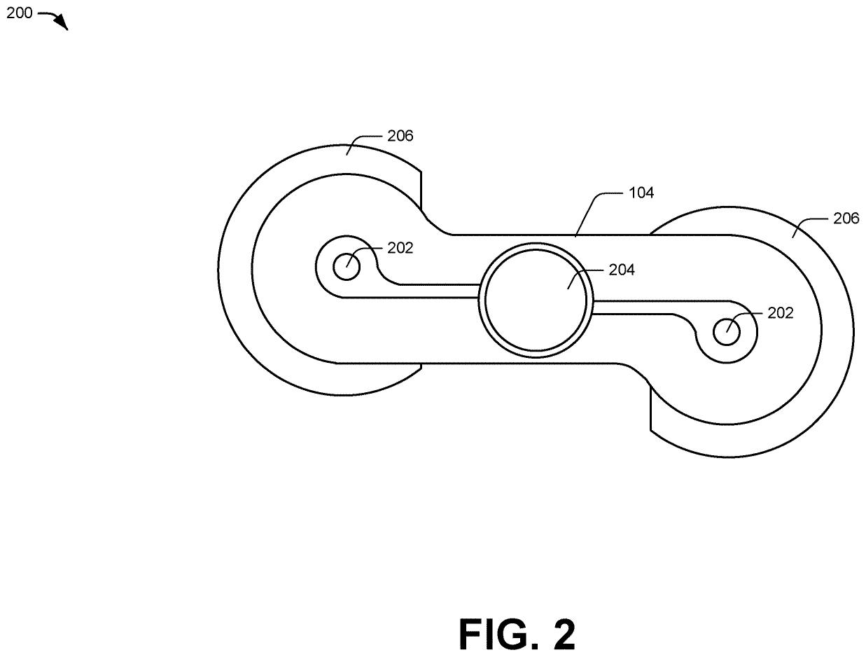

The monitoring device 104 may be configured in a variety of ways to monitor one or more aspects of the person 102. Moreover, the form factor depicted in FIGS. 1 and 2 is just one example form factor, and the form factor of the monitoring device 104 may differ in variations. It is to be appreciated that the monitoring device 104 may be configured with one or more sensors, examples of which include one or more of: a plurality of electrodes (e.g., that can be placed on the skin of the person), an accelerometer, and a pulse oximeter (e.g., to measure and record SpO2 and/or produce a photoplethysmogram of the person 102), to name just a few. It should be understood that the monitoring device 104 may be configured with any of a variety of types of sensors without departing from the described techniques.

Although the monitoring device 104 may be configured in a similar manner to monitoring devices used for clinically monitoring patients, in one or more implementations, the monitoring device 104 may be configured differently than the devices used for monitoring and/or diagnosing patients clinically. By way of example and not limitation, the monitoring device 104 may be configured as a ring, a watch, a patch, and/or a strap, to name just a few form factors. Alternatively or additionally, the monitoring device 104 may have a similar form factor as for clinical settings, but may have different functionality, such as functionality that prevents a wearer from viewing the measurements.

In one or more implementations, the monitoring device 104 may be configured to offload the measurements 108 and/or other data from the monitoring device during the course of the observation period. By way of example, the monitoring device 104 may offload the measurements 108 by transmitting them via a wired or wireless connection to an external computing device, e.g., at predetermined time intervals and/or responsive to establishing or reestablishing a connection with the computing device. In one or more implementations, the measurements 108 and/or other data from the monitoring device 104 may be compressed by the monitoring device 104 for wireless transmission, e.g., using one or more of a variety of data compression techniques. Compression of the sensor data in this way can reduce battery usage of the monitoring device 104 during the observation period and facilitate wear during assessments of physiological conditions.

To the extent that the monitoring device 104 may be configured to store the measurements 108 for an entirety of an observation period, in one or more implementations, the monitoring device 104 may be configured without wireless transmission means, e.g., without any antennae to transmit the measurements 108 wirelessly and without hardware or firmware to generate packets for such wireless transmission. Instead, the monitoring device 104 may be configured with hardware to communicate the measurements 108 via a physical, wired coupling. In such scenarios, the monitoring device 104 may be “plugged in” to extract the measurements 108 from storage of the device.

Accordingly, the monitoring device 104 may be configured with one or more ports to enable wired transmission of the measurements to an external computing device. Examples of such physical couplings may include micro universal serial bus (USB) connections, mini-USB connections, and USB-C connections, to name just a few. Although the monitoring device 104 may be configured for extraction of the measurements 108 via wired connections as discussed just above, in different scenarios, the monitoring device 104 may alternatively or additionally be configured to offload the measurements 108 over one or more wireless connections.

Once the monitoring device 104 produces the measurements 108, the measurements are provided to the analysis platform 106. As noted above, the measurements 108 may be communicated to the analysis platform 106 over wired and/or wireless connection(s).

In scenarios where the analysis platform 106 is implemented partially or entirely on the monitoring device 104, for instance, the measurements 108 may be transferred over a bus from local storage of the device to a processing system of the device. In scenarios where the monitoring device 104 is configured to generate one or more predictions 110 by processing the measurements 108, the monitoring device 104 may also be configured to provide the generated one or more predictions 110 as output, e.g., by communicating the one or more predictions 110 to an external computing device. In other scenarios, the measurements 108 may be processed by an external computing device configured to generate one or more predictions 110. For example, the measurements 108 (and/or other measurements such as accelerometer data, PPG data, SpO2 measurements, and so forth) may be processed by a smartphone associated with a user (e.g., the person 102, or an individual associated with the person 102), a smartphone or other dedicated device associated with the monitoring device 104, and/or one or more server computers at a data center or other location that can be utilized by an entity associated with the monitoring device 104, to name just a few. In other words, those other devices may implement at least a portion of the prediction system 114.

In one or more implementations, the monitoring device 104 is configured to transmit the measurements 108 to an external device over a wired connection with the external device, e.g., via USB-C or some other physical, communicative coupling. As used herein, an “external device” is meant to denote a device that is not body-worn. Here, a connector may be plugged into the monitoring device 104 or the monitoring device 104 may be inserted into an apparatus having a receptacle that interfaces with corresponding contacts of the device. The measurements 108 may then be obtained from storage of the monitoring device 104 via this wired connection, e.g., transferred over the wired connection to the external device. Such a connection may be used in scenarios where the monitoring device 104 is mailed by the person 102 after the observation period, such as to a healthcare provider, telemedicine service, provider of the monitoring device 104, or medical testing laboratory.

Alternatively or additionally, the monitoring device 104 may provide the measurements 108 to the analysis platform 106 by communicating the measurements 108 over one or more wireless connections. For example, the monitoring device 104 may wirelessly communicate the measurements 108 to external computing devices, such as a mobile phone, tablet device, laptop, smart watch, other wearable health tracker, and so on. Accordingly, the monitoring device 104 may be configured to communicate with additional (e.g., separate and/or external) devices using one or more wireless communication protocols or techniques. By way of example, the monitoring device 104 may communicate with the additional devices using one or more of Bluetooth (e.g., Bluetooth Low Energy links), near-field communication (NFC), Long Term Evolution (LTE) standards such as 5G, and so forth. The monitoring device 104 may be configured with corresponding antennae and other wireless transmission means in scenarios where the measurements 108 are communicated to an external device for processing. In those scenarios, the measurements 108 may be communicated to the analysis platform 106 in various manners, such as at predetermined time intervals (e.g., every day, every hour, or every five minutes), responsive to occurrence of some event (e.g., filling a storage buffer of the monitoring device 104), or responsive to an end of an observation period, to name just a few.

Thus, regardless of where the analysis platform 106 is implemented (e.g., at the monitoring device 104, at a smartphone associated with the person 102, or at a server device), the analysis platform 106 obtains the measurements 108 produced by the monitoring device 104. In one or more implementations, the analysis platform 106 also obtains other measurements produced by the monitoring device 104 and/or any other devices used during the observation period, e.g., a smartwatch, chest strap, and so forth (e.g., an auxiliary or separate monitoring device used in combination with the monitoring device 104). As noted above, examples of such additional measurements include but are not limited to accelerometer data, PPG measurements/signals/waveforms, and/or SpO2 measurements.

In one or more implementations, the analysis platform 106 may be implemented in whole or in part at the monitoring device 104. Alternatively, or additionally, the analysis platform 106 may be implemented in whole or in part using one or more computing devices external to the monitoring device 104, such as one or more computing devices associated with the person 102 (e.g., a mobile phone, tablet device, laptop, desktop, or smart watch) or one or more computing devices associated with a service provider (e.g., a healthcare provider, a telemedicine service, a service corresponding to the provider of the monitoring device 104, a medical testing laboratory service, and so forth). In the latter scenario, the analysis platform 106 may be implemented at least in part on one or more server devices.

In the illustrated example 100, the analysis platform 106 includes a storage device 112 and a prediction system 114. In accordance with the described techniques, the storage device 112 is configured to maintain the measurements 108 and/or other measurements or information processed by the prediction system 114 to generate the one or more predictions 110. The storage device 112 may represent one or more databases and/or other types of storage capable of storing the measurements 108 and/or other types of measurements. The storage device 112 may also store a variety of other data, such as personal information, demographic information describing the person 102, information about a healthcare provider, information about an insurance provider, payment information, prescription information, determined health indicators, account information (e.g., username and password), and so forth. The storage device 112 may also maintain data of other users of a user population.

In the illustrated example 100, the prediction system 114 represents functionality to process the measurements 108 to generate the one or more predictions 110. Alternatively, or in addition, the prediction system 114 may output one or more time sequences indicating an observation or prediction of one or more events over time. It is also to be appreciated that in variations, the prediction system 114 may output different combinations of multiple predictions.

In at least one implementation, the prediction system 114 uses machine learning and/or one or more algorithms to generate the one or more predictions 110. By way of example and not limitation, the prediction system 114 may include one or more neural networks trained based on the historical measurements and the historical outcome data of a user population. The prediction system 114 may include one or multiple machine learning models (e.g., an ensemble of models). Alternatively, or additionally, the prediction system 114 may include logic (a machine learning model and/or other types of logic) to pre-process the obtained measurements, such as to extract various cardiovascular and/or other features from the sequences of measurements. The illustrated example 100 also includes one or more predictions 110, which correspond to the output of the prediction system 114.

FIG. 2 depicts a non-limiting example 200 of a monitoring device. The illustrated example 200 depicts the monitoring device 104.

In accordance with the described techniques, the monitoring device 104 includes one or more sensors 202, examples of which include, but are not limited to, one or more pairs of electrodes, an accelerometer, a PPG sensor, temperature sensor(s), and sweat sensors, to name just a few. The monitoring device 104 may also include a transmitter 204, which may be enclosed in a housing, for example. In this example 200, the monitoring device 104 further includes one or more adhesive portions 206 configured as attachment components. In operation, the monitoring device 104 is configured to be applied to the skin via the one or more adhesive portions 206, such that the one or more sensors 202 are positioned to detect and record measurements 108, e.g., electrical activity of the heart of the person 102, for instance to produce an electrocardiogram (ECG or EKG). In at least one implementation, the monitoring device 104 may be removed by peeling the one or more adhesive portions 206 from the skin.

It is to be appreciated that the monitoring device 104 and its various components are simply one form factor, and the monitoring device 104 and its components may have different form factors without departing from the spirit or scope of the described techniques.

In one or more implementations, the monitoring device 104 may include a processor and/or memory (not shown). The monitoring device 104, by leveraging the processor, may generate the measurements 108 based on the communications with one or more sensors 202 that are indicative of some aspect of the person 102, such as one or more biological signals of the person 102. In one or more implementations, the processor further generates one or more communicable packages of data that include one or more of the measurements 108 and/or other measurements, such as accelerometer data, PPG data, and/or oxygen saturation (SpO2) measurements. Alternatively or additionally, the processor produces and/or causes storage of other data, which may be used for predicting classifications of sleep apnea.

In implementations where the monitoring device 104 is configured for wireless transmission, the transmitter 204 may transmit the measurements wirelessly as a stream of data to a computing device (e.g., the analysis platform 106). In one or more implementations, for instance, the monitoring device 104 is configured to transfer (e.g., transmit and/or receive) information (e.g., ECG and/or PPG measurements) via a Bluetooth® Low Energy (BLE) connection. Alternatively, or additionally, the monitoring device 104 may buffer the measurements 108 (e.g., in memory) and cause the transmitter 204 to transmit the buffered measurements later at various intervals, e.g., time intervals (every second, every thirty seconds, every minute, every five minutes, every hour, and so on), storage intervals (when the buffered measurements reach a threshold amount of data), and so forth.

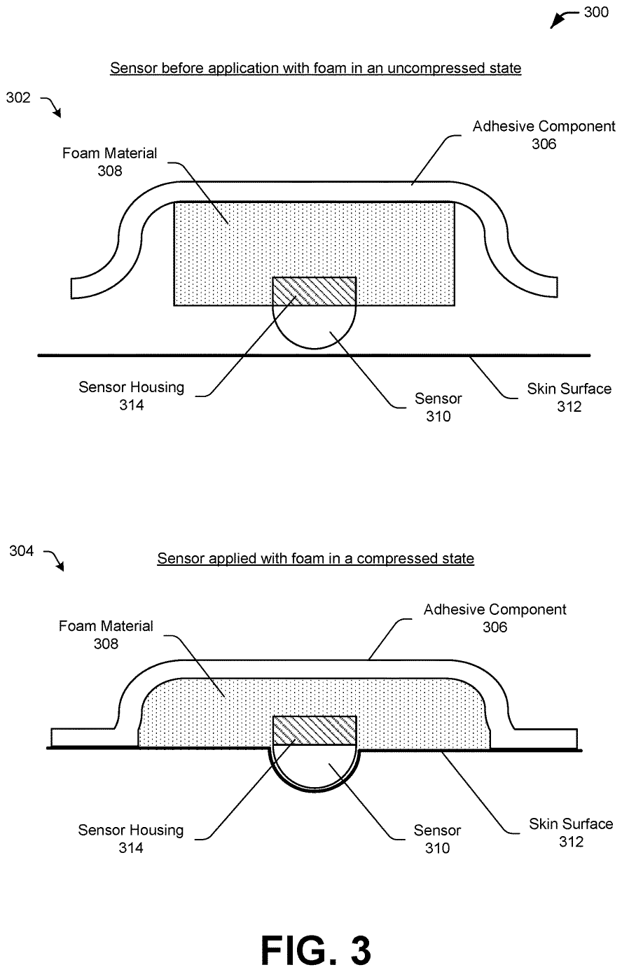

FIG. 3 illustrates an example of a sensor assembly 300 attachable to a skin surface of an individual. The example includes a first stage 302 showing the sensor assembly 300 in an uncompressed state and a second stage 304 showing the sensor assembly 300 in a compressed state when applied to a skin surface 312. In various examples, the sensor assembly 300 is the monitoring device 104 and/or includes one or more properties of the monitoring device 104.

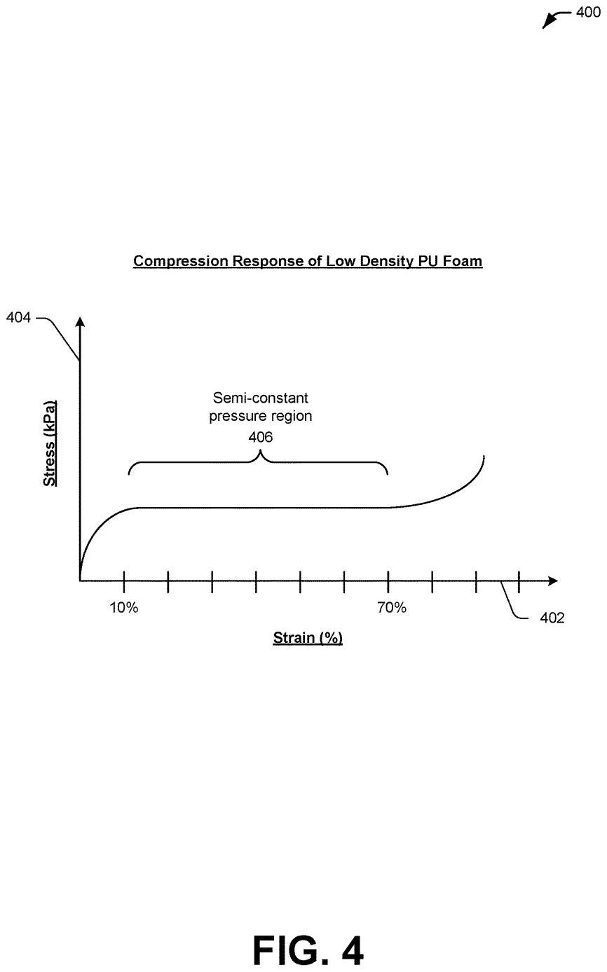

As depicted in the first stage 302, the sensor assembly 300 includes an adhesive component 306 configured to secure the sensor assembly 300 to the skin surface 312. A foam material 308 is contained within the adhesive component 306 and is disposed between the adhesive component 306 and the skin surface 312, e.g., anterior to the adhesive component 306. The foam material 308, for instance, may have a substantially uniform stress response under variable compressive strain as discussed below in more detail with respect to FIG. 4. A sensor housing 314 is disposed within the foam material 308 adjacent to the skin surface 312, e.g., anterior to the foam material 308 at an end opposite the adhesive component 306, and a sensor 310 is positioned within the sensor housing 314 substantially anterior to the foam material such that the sensor is proximal to the skin surface 312.

In the illustrated example, the adhesive component 306 extends beyond a perimeter of the foam material 308 to provide adhesion to the skin surface 312. This is by way of example and not limitation, and the adhesive component 306 may include a variety of configurations, materials, and/or structural elements to optimize sensor attachment and user comfort. In some examples, the adhesive component 306 partially or wholly encloses the foam material 308 such that at least a portion of the adhesive component 306 is disposed between the foam material 308 and the skin surface 312.

In some examples, the adhesive component 306 may include a film adhesive structure, such as a thin, flexible polymer layer with pressure-sensitive adhesive properties that conforms to skin contours while maintaining structural integrity during the observation period. The film adhesive structure may incorporate breathable materials to reduce moisture accumulation and/or skin maceration during extended wear. In one or more embodiments, the adhesive component 306 includes a multi-layer structure that incorporates one or more of a skin-friendly adhesive layer, a moisture-wicking layer, and/or a structural support layer. The adhesive component 306 may utilize various adhesives, such as but not limited to pressure-sensitive adhesives, hydrocolloid adhesives, and/or silicone-based adhesives, selected based on various factors such as wear duration, skin type compatibility, and environmental conditions.

In various implementations, the adhesive component 306 may feature a perforated or patterned design to enhance breathability and flexibility. The adhesive component 306 may incorporate regions of varying adhesive strength or elasticity to accommodate skin movement and reduce irritation. Additionally, the adhesive component 306 may include integrated channels and/or vents for moisture management and can incorporate antimicrobial properties to maintain hygiene during extended wear periods. The shape and size of the adhesive component 306 may be customized for specific body locations or sensor types, such as to include tapered edges, extended tabs for easy removal, and/or modular sections for partial replacement.

In some implementations, the adhesive component 306 may include a rigid or semi-rigid structure to provide additional support and stability to the sensor assembly. For example, the adhesive component 306 may incorporate a thin, flexible plastic or composite material frame that surrounds the foam material 308. This frame may maintain an overall shape of the sensor assembly 300 while enabling a degree of conformity to body contours of an individual. The rigid or semi-rigid structure may also serve to distribute forces evenly across the assembly, such as to reduce localized pressure points and improve overall comfort during extended wear periods, e.g., multiple days, weeks, months, etc. Additionally, this configuration may enhance durability of the sensor assembly 300, such as to insulate internal components from external stresses.

In some implementations, the adhesive component 306 may be replaced or supplemented with a non-adhesive securing mechanism, such as an armband, strap, elastic band, etc. This alternative securing mechanism may compress the foam material 308 against the skin surface 312 without directly adhering to the skin. For example, an adjustable elastic armband may encircle an arm of a user to apply pressure to the sensor assembly 300 to maintain contact between the foam material 308 and the skin surface 312. The compression provided by the armband or strap may be adjustable to accommodate different body sizes and to optimize the contact pressure for various sensor types or measurement requirements. This approach may be particularly useful for applications requiring frequent repositioning of the sensor assembly 300, for users with sensitive skin, or for scenarios where long-term adhesive wear is not desirable.

In some implementations, the foam material 308 may be combined with one or more films (e.g., a film of same or similar composition as the adhesive component 306) positioned between the foam material 308 and the sensor 310, positioned between the foam material 308 and the adhesive component 306, and/or between the foam and the skin surface 312. These films may possess particular mechanical or optical properties to enhance performance of the sensor assembly 300. For example, a film with adhesive properties may be applied to a side of the foam material 308 contacting the sensor housing 314 to secure the sensor 310 in place. Alternatively or additionally, a moisture-wicking film may be included on a side of the foam material 308 that contacts the skin surface 312 to improve comfort during extended wear.

Such films (and the foam material 308 itself) may be designed with varying optical properties, such as variable levels of transparency and/or opacity in different regions to optimize light transmission for optical sensors. One or more implementations may incorporate reflective and/or brightness-enhancing films to improve a signal quality of photoplethysmography (PPG) sensors or pulse oximeters. These films may be tailored to specific sensor types and measurement requirements, potentially improving sensor accuracy and reliability while maintaining the beneficial mechanical properties of the foam material 308.

Further, the foam material 308 is configurable in a variety of ways with various properties and/or components. The foam material 308 may include one or more materials such as viscoelastic polyurethane, memory foam, and/or other polymer-based foams with desirable mechanical properties. The foam material 308 may also incorporate composite components and/or different layers/regions with varying properties, such as a moisture-wicking layer to enhance comfort during extended wear periods. Additionally or alternatively, the foam material 308 can be infused with various additives to enhance its functionality. For example, antimicrobial agents can be incorporated into the foam material 308 such as to reduce a risk of skin infections during long-term use. The foam may also be treated with hydrophobic coatings to improve moisture resistance and maintain consistent mechanical properties in different environmental conditions.

In one or more examples, a density of the foam material 308 is tailored to provide a balance between compressibility and support. The foam material 308 may be implemented with various densities, including but not limited to one or more: low-density foams (approximately 1.5-3.0 lb/ft³) that provide gentle conformity for sensitive skin areas; medium-density foams (approximately 3.0-5.0 lb/ft³) that offer balanced support and compression characteristics for general-purpose applications; and high-density foams (approximately 5.0-7.0 lb/ft³) that provide enhanced durability and support for high-movement areas or heavier sensors. The foam material 308 may also exhibit properties such as variable recovery rates (e.g., relatively fast recovery for dynamic movements or slow recovery for sustained pressure distribution), temperature sensitivity (e.g., to maintain consistent mechanical properties across a range of skin temperatures), moisture resistance (e.g., to maintain performance in humid or sweaty conditions), and/or customized cell structures (e.g., open-cell structures for breathability or closed-cell structures for fluid resistance). These density variations and properties can be selected based on specific monitoring requirements, sensor types, body locations, individual user characteristics, and so forth.

In some examples, the foam material 308 has a variable density along one or more dimensions of the foam material 308. For example, the foam material 308 has a gradient density, such that a density of the foam material 308 changes gradually across its thickness or width. In an alternative or additional example, the foam material 308 has a layered density, and includes distinct layers with different densities that are combined. Additionally or alternatively, the foam material 308 may feature a variable radial density, with density changing from a center point to a perimeter of the foam material 308, and/or a patterned density, where particular regions of the foam have different densities to accommodate various sensor types or body contours.

In at least one example, the foam material 308 includes an anisotropic density. In this example, a density of the foam material 308 varies depending on a direction of compression, allowing for tailored responses to different types of movement or pressure. These variable density configurations support a variety of mechanical properties to suit particular monitoring scenarios, user physiologies, sensor types, and/or anatomical locations.

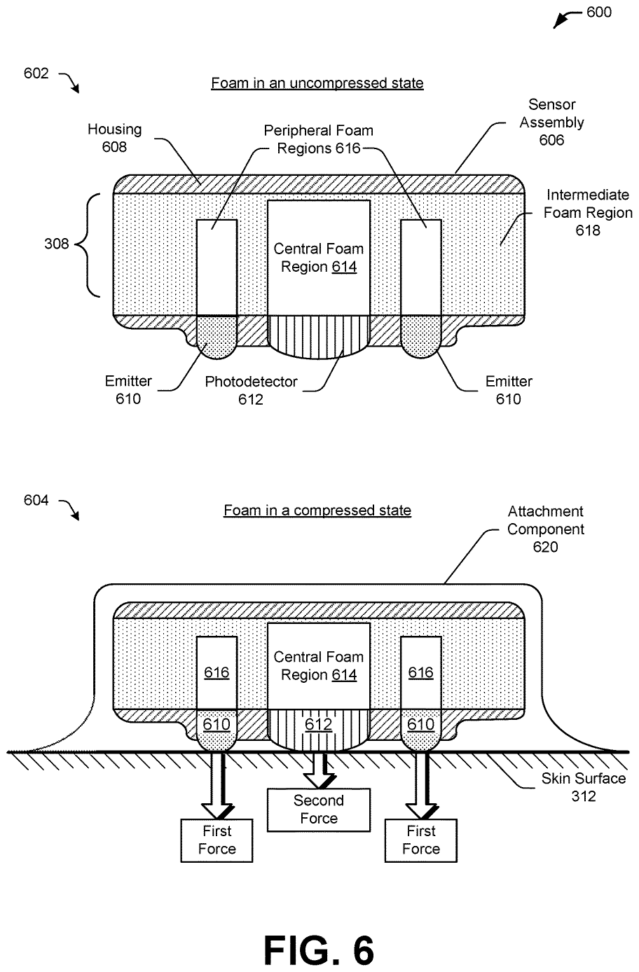

In some implementations, the foam material 308 may incorporate foam segments and/or multiple foam materials 308 with different characteristics, such as variable characteristic foam segments in regions that overlap or overlie particular sensor components. For instance, in a configuration where the sensor 310 includes a photoplethysmography (PPG) sensor array, the foam material 308 may be designed with foam segments that apply different forces/pressures to various parts of the sensor 310. This supports customized pressure application, such as a first force profile applied to light-emitting diodes (LEDs) of the PPG sensor and a second force profile applied to photodiodes of the PPG sensor. Each foam segment may be tailored in terms of density, compressibility, and other mechanical properties to optimize interaction between particular sensor components and the skin surface 312 as further described in more detail below with respect to FIG. 6.

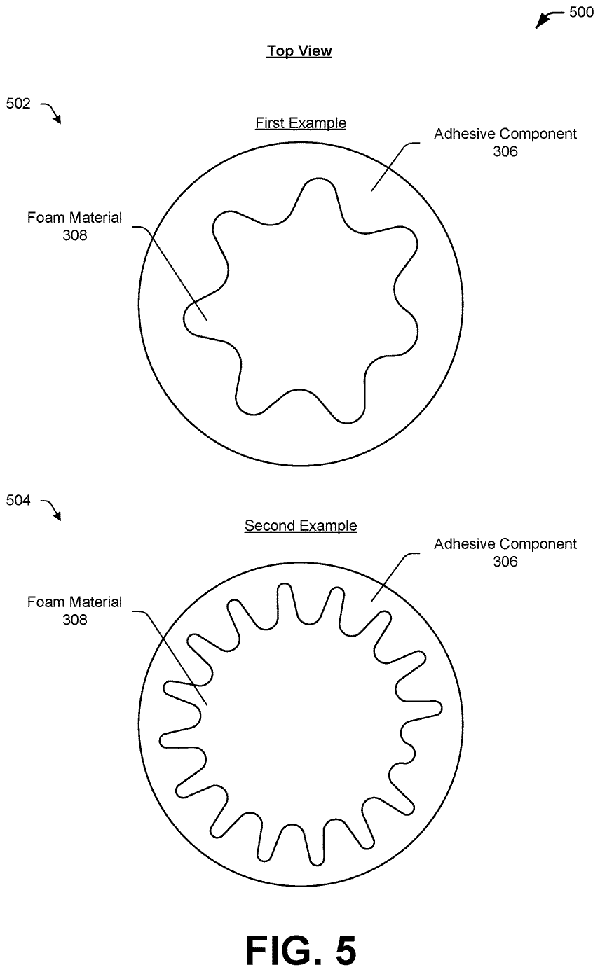

Further, a variety of shapes and thicknesses of the foam material 308 are considered. For instance, a relatively thick foam layer may be used for optical sensors that require a particular distance from the skin to be effective, while a relatively thin layer may be employed for direct contact electrodes. As further described below with respect to FIG. 5, a perimeter of the foam material 308 can be designed with particular patterns or cutouts to optimize flexibility and conformity to the skin surface while maintaining overall structural integrity.

In some implementations, the foam material 308 is formulated with various viscoelastic properties to exhibit desired time-dependent behaviors, such as controlled mechanical creep for long-term wear comfort or rapid recovery for sensors that require frequent repositioning. These properties can be fine-tuned through selection of particular foam chemistries and manufacturing processes.

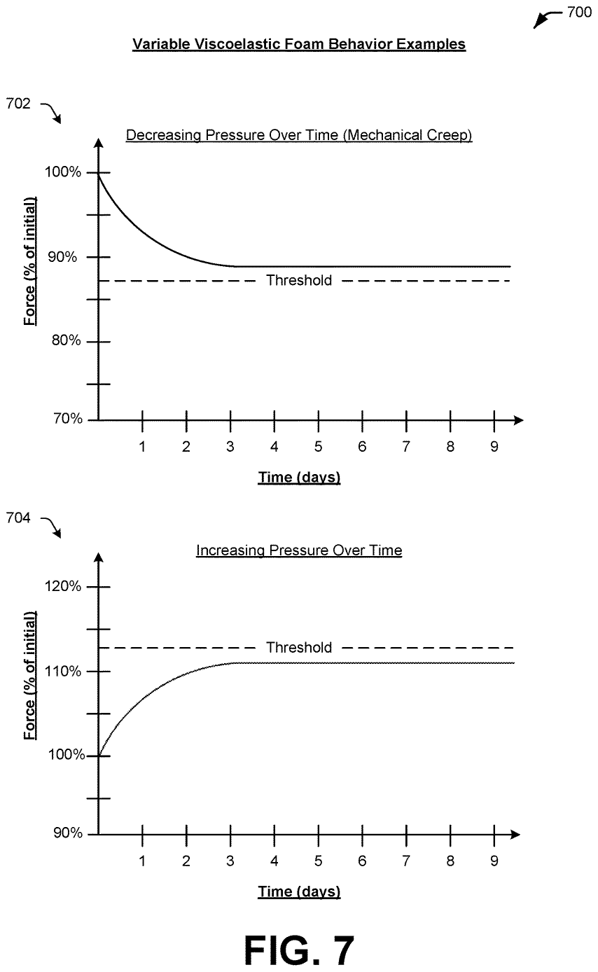

In one or more examples, the foam material 308 is designed to apply a changing pressure over time. For example, the foam material 308 can be engineered to gradually increase pressure over time, which may be beneficial for applications that benefit from progressive compression, e.g., to enhance blood flow or gradually increase sensor pressure as skin properties change during extended wear. Alternatively or additionally, the foam material 308 can be formulated to gradually decrease pressure over time, which may reduce discomfort during long-term monitoring by alleviating initial pressure points while maintaining sufficient contact for accurate measurements. This controlled pressure modulation can be achieved through specific polymer compositions that exhibit predetermined viscoelastic relaxation rates, inclusion of time-dependent additives that alter mechanical properties over predetermined periods, or through multi-layer foam structures with different time-dependent behaviors. A rate and direction of pressure change can be selected based on monitoring durations, particular body properties, sensor types used, and so forth as further described in more detail below with respect to FIG. 7.

In some implementations, the foam material 308 and/or one or more other components of the sensor assembly 300 may be formulated and/or treated to have particular visual properties that enhance a performance of the sensor 310, e.g., in an example in which the sensor 310 includes one or more optical sensors. For instance, the foam material 308 may be tinted, patterned, textured, colored, or pigmented to block a controlled amount of ambient light. This light-blocking property can be particularly beneficial for optical measurements, such as those taken by photoplethysmography (PPG) sensors or pulse oximeters. By reducing interference from external light sources, the foam material 308 may help improve a signal-to-noise ratio of optical sensor readings. The degree of light blockage can be adjusted for different sensor types and/or measurement requirements. In some cases, the foam material 308 may incorporate multiple layers with varying optical properties, allowing for fine-tuned control of light transmission and reflection.

The sensor 310 may include a variety of sensors, such as one or more of an electrode, a light sensor such as a photodiode, a light-emitting diode, one or more components of an optical sensor configured for photoplethysmography and so forth. In at least one example, the sensor 310 includes a photoplethysmography (PPG) sensor that includes a light source, e.g., a light-emitting diode, and a light sensor, e.g., a photodiode. In some examples, the sensor 310 is and/or includes properties of the sensors 202 described above. In various examples, the foam material 308 may have a thickness based in part on a type of sensor 310, such as to control a force applied by the sensor 310 to the skin surface 312. In one or more embodiments, the sensor 310 is representative of an array of sensors (e.g., two or more sensors) that are configured in a particular pattern to obtain physiological measurements. Accordingly, properties of the foam material 308 can be dependent and/or modified based on a number of sensors 310 included in the sensor assembly 300.

The sensor housing 314 in this example is configured to attach and secure the sensor 310 within the foam material 308. The sensor housing 314, for instance, may be flexible, rigid, and/or semi-rigid, and can provide structural support for the sensor 310 and maintain a position and orientation of the sensor 310 within the foam material 308. The sensor housing 314 can be designed to accommodate various sensor types and sizes, ensuring proper alignment with the skin surface 312 when the sensor assembly 300 is applied to an individual.

In some implementations, the sensor housing 314 may include features such as shock-absorbing elements to protect the sensor from impact or vibration, thermal management components to regulate sensor temperature, electrical shielding components to reduce electromagnetic interference, and so forth. Additionally, the sensor housing 314 may be designed with particular geometries or surface treatments to optimize interaction with the surrounding foam material 308, such as textured surfaces for improved adhesion and/or channels for managing moisture or airflow around the sensor 310. The sensor housing 314 may have various depths to accommodate different sensor types and sizes, ensuring proper alignment with the skin surface 312 when the sensor assembly 300 is applied to an individual.

The second stage 304 demonstrates the sensor assembly in a compressed state when applied to the skin surface 312. The foam material 308 is configured to compress between the adhesive component 306 and the skin surface 312, providing a distributed force across a contact area. This compression maintains substantially uniform pressure and contact of the sensor 310 with the skin surface 312 for a duration of an observation period.

As described above, the foam material 308 is configurable with a variety of viscoelastic properties, including but not limited to density, compression set resistance, recovery rate, temperature sensitivity, and hysteresis characteristics. Accordingly, these properties can be tailored to optimize sensor performance under compression for specific monitoring applications, body locations, individual user requirements, length of observation periods, etc. For example, the foam material 308 may be configured with relatively rapid recovery properties for applications in which frequent movement is anticipated, while the foam material 308 may be configured with a relatively slow recovery and high compression set resistance for long-term, static monitoring scenarios.

In at least one example, the observation period is an extended observation period of at least seven days. In this example, the foam material 308 is configured to exhibit mechanical creep such as to continually reduce a force applied by the sensor assembly 300 to the skin surface 312 at a particular rate during the observation period. By way of example and not limitation, the rate of force reduction is approximately 0.5% to 5% per day, depending on a particular foam formulation and thickness. In some implementations, the rate may be nonlinear, such as a rate configured to be higher during initial wear (e.g., 3-5% reduction during the first 24-48 hours) and stabilize to a lower rate (e.g., 0.5-1% per day) for a remainder of the observation period, thereby balancing initial comfort with long-term measurement stability.

In one example use case, the sensor assembly 300 is employed for continuous cardiac monitoring of patients with suspected arrhythmias. The sensor assembly 300 is applied to the chest area of an individual, with the foam material 308 conforming to body contours to maintain consistent contact between electrocardiogram (ECG) sensors of the sensor assembly 300 and the skin surface 312. Viscoelastic properties of the foam material 308 allow for comfortable wear over an extended observation period, e.g., two weeks, while ensuring consistent and suitable contact of the ECG sensors with the skin surface 312 and thus accommodate daily activities and sleep patterns of the individual while continuously collecting ECG data.

In an additional or alternative scenario, the sensor assembly 300 is utilized for long-term blood oxygen monitoring in a patient with respiratory conditions. The sensor assembly 300 is placed on the skin surface 312 of the individual (e.g., a chest region, arm, fingertip, earlobe, etc.) with the foam material 308 providing a stable mounting platform for a pulse oximeter sensor. The foam material 308 in this example is configurable with light-blocking properties such as to enhance accuracy of oxygen saturation readings by reducing interference from ambient light. It should be understood that the above-described embodiments, scenarios, and use cases are by way of example and not limitation, and various configurations of sensor assembly 300 are considered.

FIG. 4 illustrates a compression graph 400 showing example compression characteristics of the foam material 308 used in the sensor assembly 300. The compression graph 400 includes an x-axis 402 that represents strain as a percentage and a y-axis 404 that represents stress in kilopascals (kPa). As illustrated, the foam material 308 has a substantially uniform stress response under variable compressive strain.

For instance, a semi-constant pressure region 406 is indicated on the compression graph 400, which in the illustrated example occurs between approximately 10% and 70% strain. The semi-constant pressure region 406 is characterized by a relatively flat portion of the stress-strain curve, where the stress remains substantially uniform as the strain increases. This property of the foam material 308 enables the sensor assembly 300 to maintain a relatively constant pressure by applying an approximately consistent force over a contact area and thereby maintain consistent contact between the sensor 310 and the skin surface 312 during use of the sensor assembly 300 throughout an extended observation period.

Consider an example in which an individual wearing the sensor assembly 300 moves, which causes the foam to compress at different locations and magnitudes. Because of the viscoelastic properties of the foam material 308 as described herein, a pressure applied to the skin surface 312 by the sensor assembly 300 remains substantially constant rather than increasing or decreasing erratically with compression. In practice, this allows the sensor assembly 300 to maintain consistent contact pressure during activities like walking, sleeping, or exercising, without causing discomfort to an individual from pressure spikes. Additionally, this viscoelastic property enables the foam material 308 to conform to different anatomical contours while maintaining uniform sensor contact, which is beneficial when the sensor assembly 300 is worn across curved body regions.

The viscoelastic properties of the foam material 308 further reduce noise and artifacts in sensor measurements, which in turn enhances data analysis and computational efficiency. By maintaining consistent contact pressure and conforming to body movements, the foam material 308 reduces motion artifacts that arise from sensor displacement or changes in skin contact. This reduction in noise may lead to accurate and reliable data streams and reduce reliance on extensive signal processing and filtering algorithms. As a result, computational resources that would otherwise be expended on noise reduction and artifact removal may be conserved or redirected to alternative data analysis tasks. Additionally, the improved signal quality supports accurate feature extraction and pattern recognition, which results in precise physiological assessments.

The compression graph 400 shown in FIG. 4 is by way of example and not limitation, and the foam material 308 can include a variety of modular properties including, but not limited to, elastic modulus, compression set, and stress relaxation behavior. For example, the elastic modulus of the foam material 308 may range from 0.5 to 5 MPa. The compression set may be between 10% and 25%. The stress relaxation behavior may have a characteristic time of 5 to 50 seconds. These ranges are provided as non-limiting examples and may vary depending on particular requirements of the sensor assembly 300.

FIG. 5 illustrates an example 500 of different perimeter designs for a foam material 308 in a sensor assembly 300. The example 500 includes a first configuration 502 and a second configuration 504 depicted from a top view that each depict different perimeter shapes.

In the first configuration 502, a foam material 308 is shown with a non-circular perimeter, e.g., a “flower”, “wave-like”, or “scalloped” shape with various undulations, e.g., seven protrusions. A perimeter of the adhesive component 306 extends beyond the perimeter of the foam material 308, such as to attach the sensor assembly 300 to the skin surface 312. The second configuration 504 depicts an alternative design for the foam material 308, that includes a greater quantity of undulations (e.g., sixteen protrusions) around the perimeter relative to the first configuration 502.

These non-circular perimeter shapes increase a perimeter to surface area ratio relative to a circular configuration of the foam material 308 of an equivalent area. In some examples, the increased ratio reduces incidence of phenomena such as edge lifting or peel stresses and/or reduces a normal force of compression on the skin surface 312 relative to a circular configuration. The undulating patterns of both configurations thus support enhanced conformity to skin contours and improve adhesion by distributing forces evenly across the contact area.

A variety of shapes and perimeter configurations for the foam material 308 and adhesive component 306 are considered. Such configurations may include, but are not limited to, concentric circles, rectangular shapes, star-shaped configurations, hexagonal patterns, scalloped edges, triangular extensions, oval formations, rectangular designs with rounded corners, asymmetrical patterns tailored to specific anatomical regions, various polygonal shapes, and so forth. In some examples, the perimeter shape of the foam material 308 and adhesive component 306 interface are configured to reduce effects of medical adhesive-related skin injuries (MARSI). For instance, an increased edge length provided by these non-circular designs depicted in the first configuration 502 and the second configuration 504 may distribute adhesive forces more evenly, potentially reducing stress on the skin during wear and removal.

Accordingly, the techniques described herein provide improved comfort, data quality, and long-term wearability for various physiological monitoring applications relative to conventional techniques. By leveraging viscoelastic foam properties and optimized design elements, the sensor assembly 300 provides a versatile solution for continuous health monitoring across diverse patient populations and use cases.

FIG. 6 illustrates an example 600 of an uncompressed state 602 and a compressed state 604 of a sensor assembly 606 that includes multiple foam regions. The example 600 depicts cross-sectional views of the sensor assembly 606 to demonstrate behavior of various regions of a foam material 308 during application of the sensor assembly 606 to the skin surface 312. The sensor assembly 606, for instance, is representative of one or more components of a monitoring device 104.

In this example, the sensor assembly 606 is depicted to include a housing 608, e.g., a rigid structure or a semi-rigid structure to partially or wholly enclose or contain the foam material 308 and/or various components of the sensor assembly 606. The housing 608 may be configured to orient and position components of the sensor assembly 606, such as to maintain alignment of components relative to one another and/or the skin surface 312. For example, the housing 608 may secure hardware components, including sensors, circuit boards, wiring, and/or connectors, while providing structural support to protect internal elements from external forces during wear.

The sensor assembly 606 in this example further includes optical components positioned at a bottom region of the assembly, including two emitters 610 located on either side of a photodetector 612 positioned centrally between the emitters 610. The emitters 610, for instance, may include light-emitting diode (LED) sensors for optical measurements, and the photodetector 612 may include a photodiode to form a photoplethysmography (PPG) sensor array.

In an example, the PPG sensor array functions by emitting light from the emitters 610 into an adjacent skin surface 312, where the light penetrates underlying tissue and blood vessels. A portion of the emitted light is absorbed by blood in the vessels, while another portion is reflected back toward the photodetector 612. As blood volume in the vessels changes with each heartbeat, the amount of light absorbed varies correspondingly, causing fluctuations in the reflected light detected by the photodetector 612. The photodetector 612 converts light intensity variations into electrical signals that can be processed to determine physiological parameters such as heart rate, heart rate variability, and blood oxygen saturation.

In various scenarios, the emitters 610 and the photodetector 612 have different mechanical conditions, e.g., contact pressure requirements, for effective signal acquisition. For instance, the emitters 610 may benefit from a particular contact pressure with the skin surface 312 that ensures efficient light coupling into the tissue without blanching the skin surface 312 and altering local blood perfusion. The photodetector 612 may perform with increased accuracy at a different contact pressure condition, e.g., a relatively lower contact pressure, to maintain stable optical coupling while minimizing motion artifacts that interfere with detection of reflected light signals. Thus, conventional sensor mounting approaches that apply uniform pressure across sensor components can result in suboptimal signal quality for one or more components of a multi-element sensor array.

Accordingly, it can be desirable to apply different mechanical conditions to sensors 202 of the monitoring device 104, such as the emitters 610 and the photodetector 612 in the illustrated example. To do so, the foam material 308 can include regions with variable geometric, viscoelastic, density, compressibility, optical, and/or mechanical properties. In the example 600, the foam material 308 is organized into distinct regions that include peripheral foam regions 616 in line with and adjacent to the emitters 610, a central foam region 614 positioned in line with and adjacent to the photodetector 612, and an intermediate foam region 618 situated between the peripheral foam regions 616 and the central foam region 614. Each region of the foam material 308 may have different properties configured to impart a particular effect on respective components of the sensor assembly 606.

For instance, the central foam region 614 may have a first property and the peripheral foam regions 616 may have a second property different from the first property, e.g., one or more variable densities, compressibilities, optical properties, geometries, and so forth. As depicted in FIG. 6, the peripheral foam regions 616 are positioned in line with and adjacent to the emitters 610, while the central foam region 614 is positioned in line with and adjacent to the photodetector 612. Accordingly, when the foam material 308 is compressed, the mechanical behavior of the emitter 610 is based on properties of the peripheral foam regions 616, while the mechanical behavior of the photodetector 612 is based on properties of the central foam region 614.

The intermediate foam region 618 is positioned between the peripheral foam regions 616 and the central foam region 614, such as to provide a transition zone that accommodates the different mechanical behaviors of adjacent regions while maintaining structural integrity of the foam material 308. In various examples, the sensor assembly 606 includes one or more rigid or non-rigid dividers between different foam regions.

In the compressed state 604, the sensor assembly 606 is shown applied to the skin surface 312. The attachment component 620 is positioned to at least partially enclose the housing 608 of the sensor assembly 606 and to secure the sensor assembly 606 to the skin surface 312. As described in more detail above, the attachment component 620 may include one or more of an adhesive component, rigid or semirigid components, an armband, a strap, an elastic band, and so forth. When coupled to the housing 608 and the skin surface 312, the attachment component 620 is configured to apply one or more compressive forces, e.g., a “downward” force, such as to compress the foam material 308 between the attachment component 620 and the skin surface 312.

Due to the variable properties of the different regions of the foam material 308, mechanical behaviors of the emitters 610 and photodetector 612 are controllable to enhance performance of each respective component. For example, density variations between foam regions are configurable to control a magnitude of force applied to each component, such that relatively high-density regions apply a relatively greater force to an associated component and relatively low-density regions apply a relatively reduced force to an associated component. Additionally or alternatively, the respective foam regions are configurable with various compressibility properties to control how a particular region responds to external pressure, recovery rate properties to influence how quickly a particular region returns to an original shape after compression (such as to reduce motion artifact reduction for different sensor types), with geometric/spatial properties such as thickness and cross-sectional area of each foam region, such as to modulate a force distribution on components, and so forth.

In the illustrated example of FIG. 6, the peripheral foam regions 616 are configured with properties that apply a first force profile to the emitters 610, such that the emitters 610 apply a first contact force to the skin surface 312. This first contact force may be configured to ensure efficient light transmission into the tissue without blanching the skin surface 312. The central foam region 614 is configured with different properties that apply a second force profile to the photodetector 612, such that the photodetector 612 applies a second contact force, e.g., a force with a relatively smaller magnitude, to the skin surface 312. This reduced force at the photodetector 612 maintains stable optical coupling while reducing incidence of irritation to a person 102.

Further, the central foam region 614 is depicted to have a larger cross-sectional area relative to the peripheral foam regions 616, such as to distribute force more evenly across the photodetector 612 and maintain stable optical coupling while minimizing motion artifacts in the detected signal. The foam regions may also have different recovery rates, such as to accommodate movement of the individual during the observation period. In the illustrated example, the peripheral foam regions 616 adjacent to the emitters 610 exhibit a relatively slow recovery rate, such as to maintain stable light coupling during movement, while the central foam region 614 over the photodetector 612 exhibits a relatively fast recovery rate to adapt to movement and reduce motion artifacts in the detected signal.

Accordingly, the techniques, components, and assemblies described herein leverage various configurations of the viscoelastic foam material 308 to optimize interaction between particular sensor components and the skin surface 312. Further, the variable properties of the foam regions enable the sensor assembly 606 to adapt to motion of an individual during an observation period, by providing component specific responses to various external forces.

FIG. 7 illustrates examples 700 of variable viscoelastic foam behavior over time. As described above, in some examples, the foam material 308 may be configured to change pressure characteristics (e.g., a pressure applied to one or more sensors 202 to impact contact force with a skin surface 312) over time in a known manner for various use cases. The examples 700 include a first graph 702 and a second graph 704 that depict different time-dependent pressure behaviors achievable through engineering of foam material properties. In the graphs, a y-axis represents force as a percentage of initial force, and an x-axis represents time in days.

The first graph 702 depicts an example in which the foam material 308 is configured to decrease pressure over time due to controlled mechanical creep of the foam material 308. In one or more embodiments, the foam material 308 is able to recover form, e.g., the foam material 308 includes one or more suitable shape-memory polymers, and/or is expandable such as due to an environmental or physical action or stimuli. The first graph 702 shows a curve that begins at 100% initial force and gradually decreases over approximately nine days, with a threshold line indicated at approximately 87% of the initial force. In this example, the rate of force reduction is nonlinear, such as a rate configured to be higher during initial wear (e.g., 3.0-5.0% reduction during the first 24-48 hours) and stabilize to a relatively lower rate (e.g., 0.0-1.0% per day) for a remainder of the observation period before reaching a threshold.

Such decreasing pressure behavior may be desirable for applications where prolonged sensor contact is anticipated, such as to provide a relatively larger force during the first few days of an observation period to ensure proper adhesion of the monitoring device 104 to the person 102, with a reduction in applied force to reduce a risk of pressure-related skin injuries, such as pressure ulcers or medical adhesive-related skin injuries (MARSI), during extended monitoring periods. This behavior may be achieved by selecting foam compositions with particular viscoelastic properties that exhibit time-dependent stress relaxation, such as polyurethane foams with particular cell structures and/or densities that promote gradual deformation under sustained compression.

The second graph 704 depicts an example in which the foam material 308 is configured to increase pressure over time. The second graph 704 shows a curve that begins at approximately 100% force and increases over time, leveling off at approximately 110% of initial force, with a threshold line indicated at approximately 110%. In this example, the rate of force increase may be configured to be higher during initial wear (e.g., 3.0-5.0% increase during the first 24-48 hours) and stabilize to a relatively lower rate (e.g., 0.0-1.0% per day) for a remainder of the observation period to reach the threshold.

Such increasing pressure behavior may be desirable for applications where initial sensor application with a relatively low contact force is preferable, such as to allow for patient comfort during initial placement, with increased contact pressure to improve signal quality as the sensor “settles” into position on the skin surface 312. As such, the examples 700 demonstrate how foam material properties may be engineered to exhibit different time-dependent pressure behaviors for various monitoring applications. By selecting or formulating foam materials with particular compositions, cell structures, densities, or responsive additives, the time-dependent force profile of the sensor assembly 300 may be tailored to match requirements of specific monitoring scenarios.

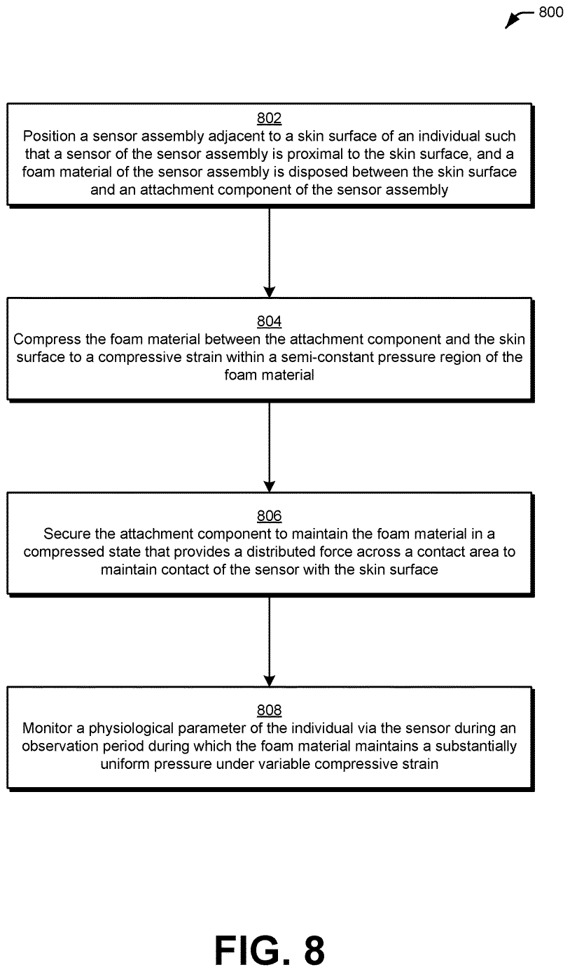

FIG. 8 depicts a flow diagram of a method 800 in an example implementation that is performable to apply a sensor assembly to a skin surface of an individual.

To begin in this example, a sensor assembly is positioned adjacent to a skin surface of an individual such that a sensor of the sensor assembly is proximal to the skin surface, and a foam material of the sensor assembly is disposed between the skin surface and an attachment component of the sensor assembly (block 802). In various examples, the sensor assembly is positioned such that the sensor is oriented toward the skin surface 312 to enable detection of physiological parameters of the individual. The foam material 308 is disposed between the attachment component and the skin surface 312 to provide a cushion layer that distributes force across a contact area.

The foam material is then compressed between the attachment component and the skin surface to a compressive strain within a semi-constant pressure region of the foam material (block 804). As described in more detail above, the semi-constant pressure region may correspond to a range between approximately 10% and 70% compressive strain. The sensor assembly can be applied to press the sensor into the surface of the skin with a desired contact force while the foam material compresses to the 10-70% strain region. The compression of the foam material 308 within the semi-constant pressure region enables the foam material 308 to maintain a relatively uniform stress response despite variations in compressive strain that may occur during movement of the individual.

The attachment component is secured to maintain the foam material in a compressed state that provides a distributed force across a contact area to maintain contact of the sensor with the skin surface (block 806). The attachment component, for instance, may include one or more adhesive components 306 that adhere to the skin surface 312 around a perimeter of the foam material 308. In some cases, the attachment component may include one or more non-adhesive components, rigid or non-rigid components, such as an armband, a strap, or an elastic band. The distributed force provided by the compressed foam material 308 maintains contact between the sensor 310 and the skin surface 312 throughout an observation period. The foam material 308 allows for adequate intrusion depth and pressure on the skin surface 312 for reduced noise artifacts while reducing occurrence of pressure sores and ulcers.

A physiological parameter of the individual is monitored via the sensor during an observation period during which the foam material maintains a substantially uniform pressure under variable compressive strain (block 808). In various examples, the sensor 310 may include one or more of an electrode, a photodiode, or a light-emitting diode to detect physiological parameters such as electrocardiography data, pulse oximetry data, or other measurements. The foam material 308 maintains consistent contact between the sensor 310 and the skin surface 312 throughout the observation period, which may extend for multiple days. In some cases, the foam material 308 exhibits time-dependent behaviors during extended observation periods, such as to modulate a contact force applied by the sensor assembly to the skin surface 312 over time.

Accordingly, the techniques described herein provide improved comfort, data quality, and long-term wearability for various physiological monitoring applications relative to conventional techniques. By leveraging viscoelastic foam properties and optimized design elements, the sensor assembly 300 provides a versatile solution for continuous health monitoring across diverse patient populations and use cases.

It should be understood that many variations are possible based on the disclosure herein. Although features and elements are described above in particular combinations, each feature or element is usable alone without the other features and elements or in various combinations with or without other features and elements.

What is claimed is:

1. A sensor assembly attachable to a skin surface of an individual, the sensor assembly comprising:
an adhesive component configured to secure the sensor assembly to the skin surface;
a foam material at least partially contained within the adhesive component to be disposed between the adhesive component and the skin surface, the foam material having a substantially uniform stress response under variable compressive strain;
a sensor housing region disposed within the foam material; and
a sensor positioned within the sensor housing region, the foam material configured to compress between the adhesive component and the skin surface to provide a distributed force across a contact area to maintain contact of the sensor with the skin surface for a duration of an observation period.

2. The sensor assembly of claim 1, wherein the foam material is a viscoelastic polyurethane foam.

3. The sensor assembly of claim 1, wherein a compression response of the foam material exhibits a semi-constant pressure region between 10% and 70% compressive strain.

4. The sensor assembly of claim 1, wherein the adhesive component extends beyond a perimeter of the foam material to provide adhesion to the skin surface.

5. The sensor assembly of claim 1, wherein the sensor includes one or more of an electrode, or an optical sensor configured for photoplethysmography, the optical sensor including one or more of a photodiode or a light-emitting diode.

6. The sensor assembly of claim 1, wherein the adhesive component includes a film adhesive structure.

7. The sensor assembly of claim 1, wherein the foam material has a thickness based in part on a type of sensor to control a force applied by the sensor to the skin surface.

8. The sensor assembly of claim 1, wherein the foam material has a non-circular perimeter shape to increase a perimeter to surface area ratio relative to a circular configuration of the foam material of an equivalent area to reduce edge lifting or peel stresses.

9. The sensor assembly of claim 1, wherein the observation period is at least seven days, and the foam material is configured to exhibit mechanical creep to continually reduce a force applied by the sensor assembly to the skin surface at a particular rate during the observation period.

10. A foam material for use in a sensor assembly attachable to a skin surface of an individual, the foam material comprising:
a viscoelastic composition having a substantially uniform stress response under variable compressive strain;
a sensor housing region disposed within the foam material and configured to receive a sensor; and
a compression response that exhibits a semi-constant pressure region across a range of compressive strain, the foam material configured to compress between an attachment component of the sensor assembly and the skin surface to provide a distributed force across a contact area.

11. The foam material of claim 10, wherein the foam material is a viscoelastic polyurethane foam and the semi-constant pressure region is between 10% and 70% compressive strain.

12. The foam material of claim 10, wherein the foam material has a variable density along one or more dimensions of the foam material.

13. The foam material of claim 12, wherein the variable density includes one or more of a gradient density, a layered density, a variable radial density, a patterned density, or an anisotropic density.

14. The foam material of claim 10, wherein the foam material is tinted to block a controlled amount of ambient light.

15. The foam material of claim 10, wherein the foam material has a non-circular perimeter shape to increase a perimeter to surface area ratio relative to a circular configuration of the foam material of an equivalent area to reduce one or more of edge lifting, peel stresses, or a normal force of compression on the skin surface relative to the circular configuration.

16. A sensor assembly attachable to a skin surface of an individual, the sensor assembly comprising:
an attachment component configured to secure the sensor assembly to the skin surface;
a foam material having a substantially uniform stress response under variable compressive strain that includes a first region having a first property and a second region having a second property different from the first property; and
a sensor configured to be disposed adjacent to the skin surface, the foam material configured to compress between the attachment component and the skin surface to maintain contact of the sensor with the skin surface during an observation period.

17. The sensor assembly of claim 16, wherein the first property and the second property include one or more variable densities, compressibilities, optical properties, or geometries.

18. The sensor assembly of claim 17, wherein the sensor includes a photoplethysmography (PPG) sensor array including one or more light-emitting diodes and one or more photodiodes, and wherein the first region overlies the one or more light-emitting diodes and the second region overlies the one or more photodiodes.

19. The sensor assembly of claim 18, wherein the first region applies a first force to the one or more light-emitting diodes when compressed and the second region applies a second force to the one or more photodiodes when compressed, the first force different than the second force.

20. The sensor assembly of claim 16, wherein the attachment component includes one or more of an adhesive component, an armband, a strap, or an elastic band.
